(19) United States
(12) Patent Application Publication
(10) Pub. No.: US 2015/0202340 A1
(43) Pub. Date: Jul. 23, 2015

(54) Scent Dispenser System

(71) Applicant: Earth-Kind, Inc., Bismarck, ND (US)

(72) Inventor: Kari G. Warberg Block, Bismarck, ND (US)

(73) Assignee: Earth-Kind, Inc., Bismarck, ND (US)

(21) Appl. No.: 14/162,119

(22) Filed: Jan. 23, 2014

(51) Int. Cl. A61L 9/12

(52) U.S. Cl. CPC A61L 9/12

(57) ABSTRACT

An scent dispenser housing system for housing an air freshener within a hanging position or a supported position. The scent dispenser housing system generally includes a housing having a base member having a plurality of base vents and a cover member having a plurality of cover vents removably connected to the base member to form an interior cavity that receives a fragrance dispenser. A connecting member is removably connected to the housing to allow the housing to be hung from an object when attached to the housing or removed to allow for the housing to be positioned upon a support surface such as a table.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to an air fragrance dispenser and more specifically it relates to an scent dispenser housing system for housing an air freshener within a hanging position or a supported position.

2. Description of the Related Art

Any discussion of the related art throughout the specification should in no way be considered as an admission that such related art is widely known or forms part of common general knowledge in the field.

Conventional air fresheners are typically comprised of either a hanging structure or a supported structure. Air fresheners that are comprised of a hanging structure typically have a hook or loop connected to a housing structure that extends upwardly for removable attachment to a fastener or other object. Air fresheners that are comprised of a supported structure have a support base that is supported upon an upper surface of a table or other platform. The housings for both hanging and supported air fresheners typically are comprised of a non-openable structure.

Conventional air fresheners are not designed to be used in either a hanging position or a supported position since they are designed for only one position. Hence, a consumer must purchase a specific type of air freshener for their initial intended usage (e.g. supported position upon a table) and are thereafter limited to that initial usage thereafter. In addition, conventional air fresheners within housings also typically do not provide for an easy to open and reclose housing structure to allow for replacement of an air freshener material within the housing.

Because of the inherent problems with the related art, there is a need for a new and improved scent dispenser housing system for housing an air freshener within a hanging position or a supported position.

### BRIEF SUMMARY OF THE INVENTION

The invention generally relates to an air freshener which is comprised of a housing having a base member having a plurality of base vents and a cover member having a plurality of cover vents removably connected to the base member to form an interior cavity that receives a fragrance dispenser. A connecting member is removably connected to the housing to allow the housing to be hung from an object when attached to the housing or removed to allow for the housing to be positioned upon a support surface such as a table.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1a is a front upper perspective view of the present invention with the connecting member connected to the housing thereby allowing the housing to be positioned in a hanging position upon an object such as a fastener.

FIG. 1b is a front upper perspective view of the present invention with the connecting member removed from the housing thereby allowing the housing to be positioned in a supported position upon an upper surface of a table.

FIG. 2 is a rear upper perspective view of the present invention.

FIG. 3 is a front view of the present invention.

FIG. 4 is a rear view of the present invention.

FIG. 5 is a left side view of the present invention.

FIG. 6 is a right side view of the present invention.

FIG. 7 is a bottom view of the present invention.

FIG. 8 is a top view of the present invention.

FIG. 9 is an exploded front upper perspective view of the present invention.

FIG. 10 is an exploded rear upper perspective view of the present invention.

FIG. 11 is a cutaway view taken along line 11-11 of FIG. 1a.

FIG. 12 is a cutaway view taken along line 12-12 of FIG. 7.

FIG. 13 is an upper perspective view of the present invention hanging from a fastener.

FIG. 14 is an upper perspective view of the present invention supported upon the upper surface of a table.

### DETAILED DESCRIPTION OF THE INVENTION

### A. Overview.

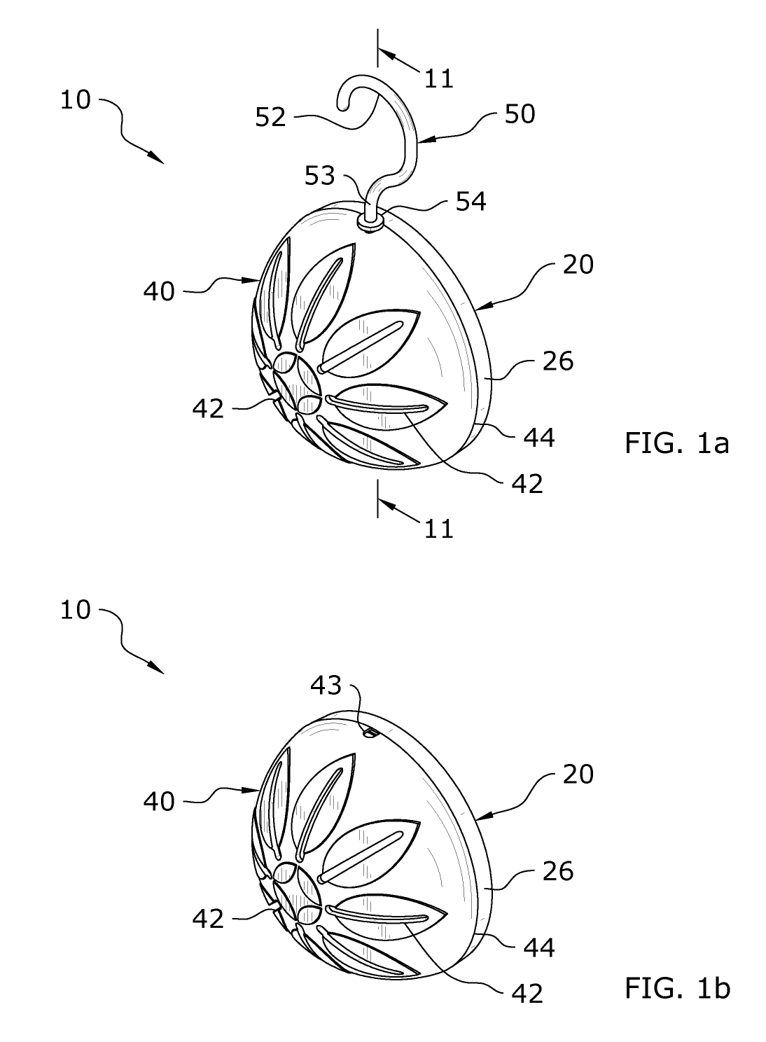

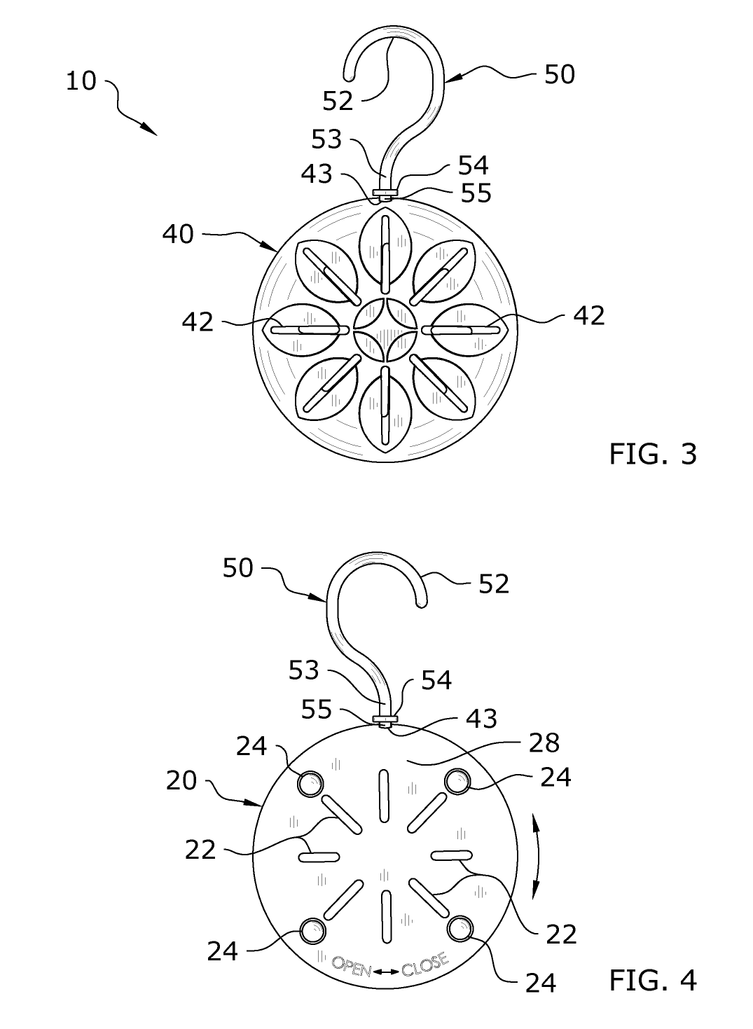

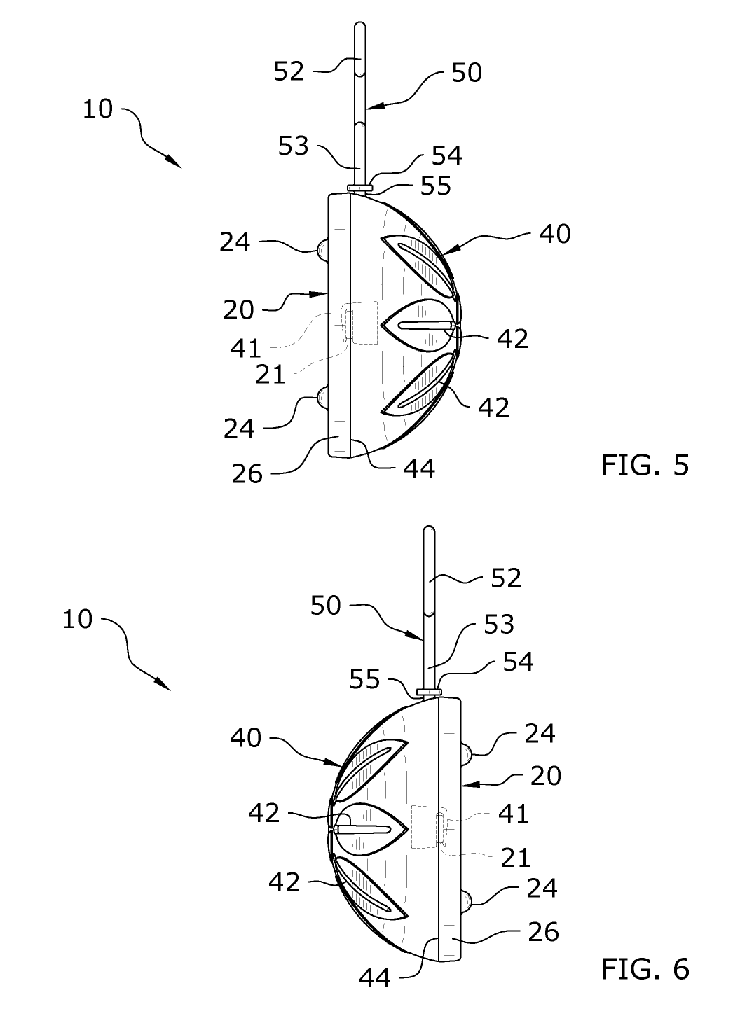

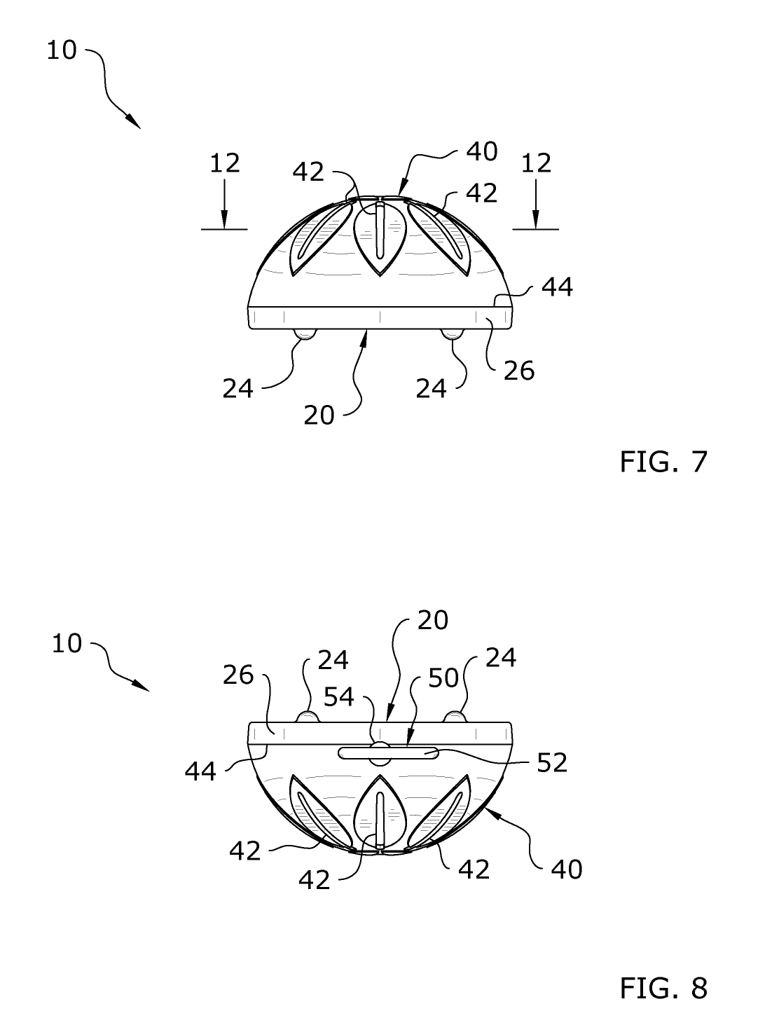

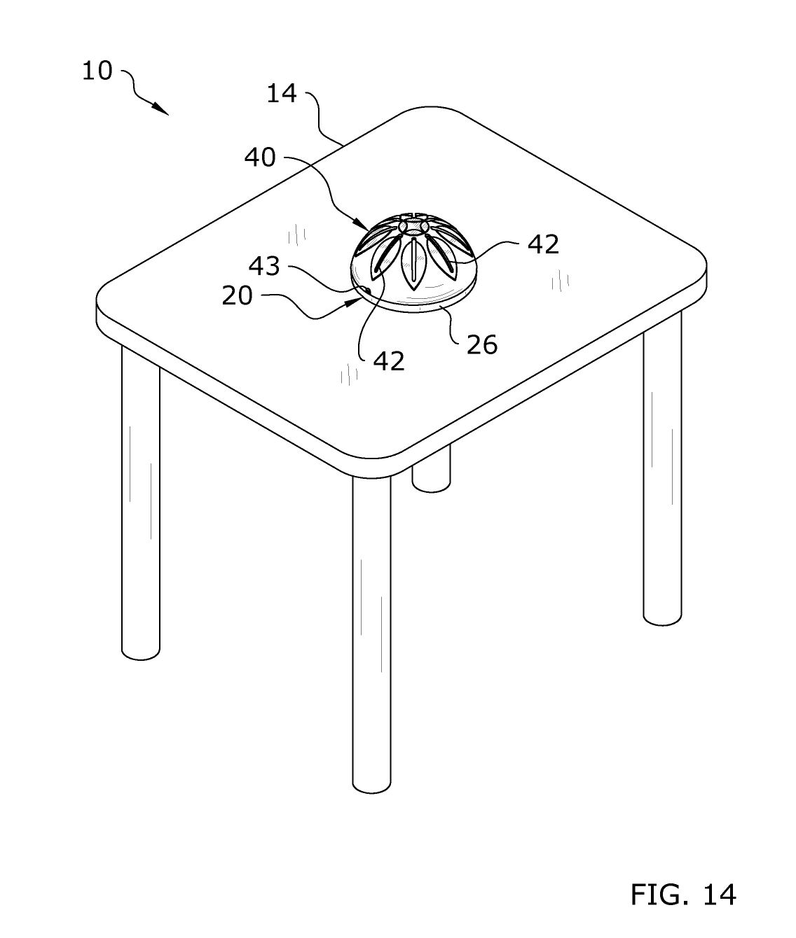

Turning now descriptively to the drawings, in which similar reference characters denote similar elements throughout the several views, FIGS. 1 through 14 illustrate an scent dispenser housing system 10, which comprises a housing having a base member 20 having a plurality of base vents 22 and a cover member 40 having a plurality of cover vents 42 removably connected to the base member 20 to form an interior cavity that receives a fragrance dispenser 30. A connecting member 50 is removably connected to the housing to allow the housing to be hung from an object when attached to the housing or removed to allow for the housing to be positioned upon a support surface such as a table 14.

### B. Housing.

1. Overview.

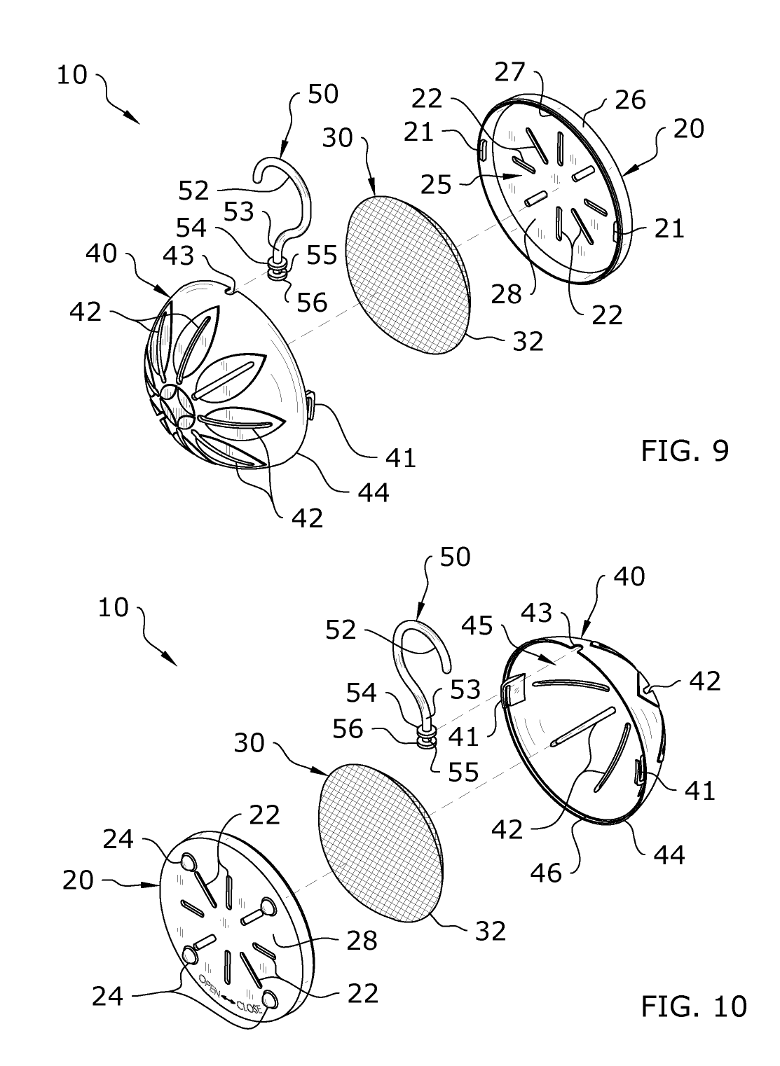

An exemplary housing is illustrated in FIGS. 1a through 14 of the drawings. The housing is preferably comprised of a circular shaped structure, however, various other configurations may be used to construct the housing. The housing is preferably comprised of a base member 20 and a cover member 40 as best illustrated in FIGS. 9 and 10 of the drawings that are separable from and attachable to one another to allow for replacement of a fragrance dispenser 30 from within an interior cavity of the housing.

The housing is preferably constructed of a plastic material such as biodegradable plastic (e.g. polyactic acid derived from renewable resources). The housing may be constructed of various other types of materials (e.g. metal, wood and the like). The housing may have various sizes such as but not limited to diameters ranging between 4 inches to 6 inches. The thickness (back to front) of the housing may be but is not limited to the range of 1 inch to 3 inches.

The fragrance dispenser 30 may be comprised of any dispenser capable of dispensing a fragrance. However, it is preferable that the fragrance dispenser 30 be comprised of a vented pouch 32 that retains material within impregnated with a fragrance oil. U.S. Pat. No. 6,337,081 to Kari Warberg illustrates an exemplary fragrance dispenser 30 and is incorporated by reference herein. The vented pouch 32 is preferably comprised of a material that is capable of retaining the scented material but allows for airflow through thereby allowing the fragrance to be emitted from the interior of the housing externally.

2. Cover Member.

The cover member 40 preferably has a dome shape as illustrated in FIGS. 1a, 1b, 5 and 6 of the drawings. The cover member 40 further preferably is comprised of a hollow dome shaped structure as best illustrated in FIGS. 9 and 10 of the drawings. The cover member 40 is formed to connect to the base member 20 to form the housing having the appearance of a unitary structure. The cover member 40 defines a cover cavity 45 as best illustrated in FIG. 10 of the drawings. The cover member 40 is removably connected to the base member 20 to form an interior cavity within the housing that receives a fragrance dispenser 30 and wherein air is allowed to freely flow through the at least one cover vent through the interior cavity then through the at least one base vent. The cover member 40 has a convex outer wall with an outer rim 44 along the perimeter thereof.

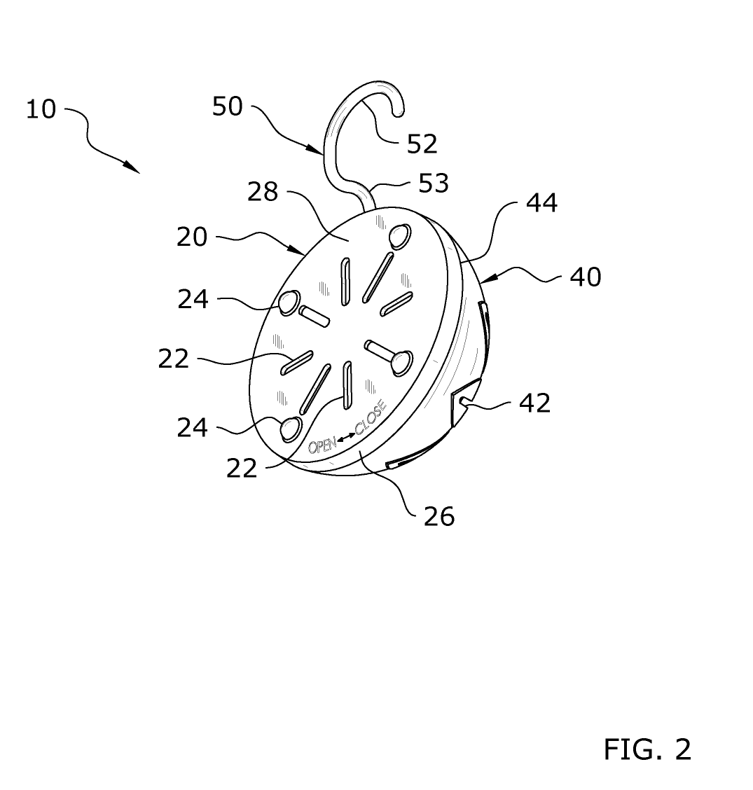

The cover member 40 is preferably rotatably connected to and rotatably removed from the base member 20 utilizing connectors attached to the cover member 40 and the base member 20 that removably mate with one another. FIG. 2 illustrates instructions attached or embedded within the outer surface of the wall 28 of the base member 20 indicating to the user which direction to rotate to either open or close the housing. When the housing is opened, the user is able to remove an exhausted fragrance dispenser 30 and insert a new fragrance dispenser 30.

3. Base Member.

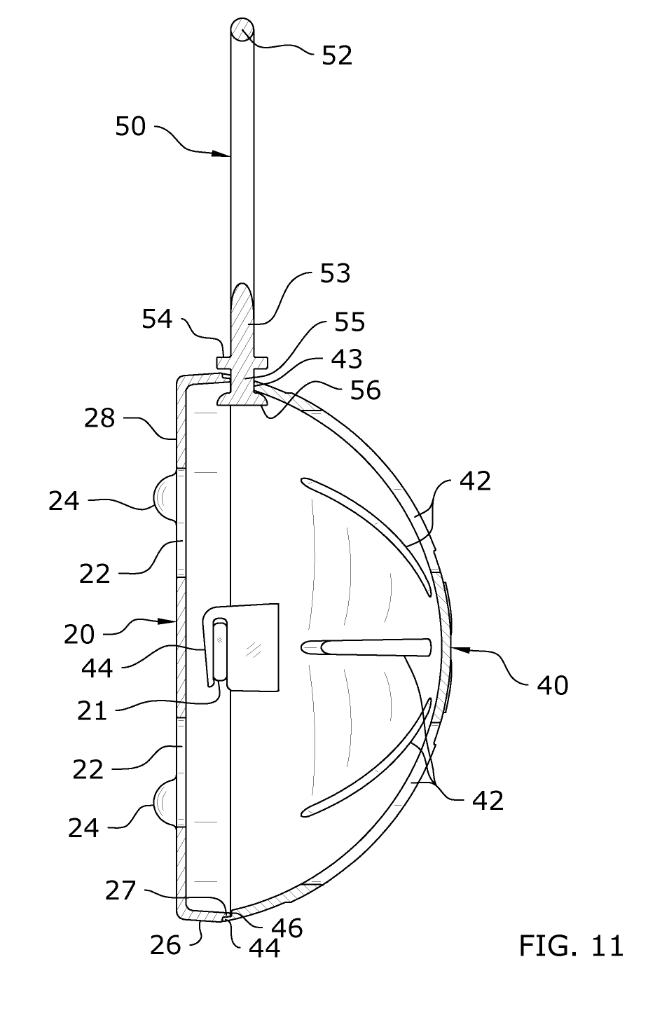

The base member 20 includes a wall 28 and a base rim 26 that extends forwardly and outwardly from the base member 20 as best illustrated in FIGS. 9 and 11 of the drawings. The wall 28 and the base rim 26 are preferably circular shaped and have a size equal to the size of the cover member 40. The wall 28 and the base rim 26 define a base cavity 25, wherein the base cavity 25 and the cover cavity 45 together form the interior cavity of the housing when the cover member 40 is connected to the base member 20.

The base rim 26 preferably includes a base recessed portion 27 within an outer perimeter of the base rim 26 to receive a corresponding shaped outer perimeter of the cover member 40 as illustrated in FIG. 9 of the drawings. The corresponding shaped outer perimeter of the cover member 40 includes a cover recessed portion 46, wherein the cover recessed portion 46 is on a side of the cover member 40 opposite of a side of the base rim 26 of the base member 20 as illustrated in FIG. 10 of the drawings. The base recessed portion 27 extends along the entire perimeter of the base rim 26 and the cover recessed portion 46 extends along the entire perimeter of the cover member 40 as illustrated in FIGS. 9 and 10 of the drawings. The depth of the base recessed portion 27 is preferably equal to the depth of the cover recessed portion 46 to form a snug fit between the cover member 40 and the base member 20.

### C. Base Vents.

As illustrated in FIGS. 2, 4, 9 and 10 of the drawings, the base member 20 includes at least one base vent and preferably includes a plurality of base vents 22. The base vents 22 allow for airflow through the base member 20 to allow for the fragrance emitted into the air from the fragrance dispenser 30 to be emitted externally of the housing. The plurality of base vents 22 are preferably comprised of a plurality of slots that extend radially outwardly from a central portion of the wall 28 as best illustrated in FIG. 4 of the drawings.

### D. Cover Vents.

As illustrated in FIGS. 1a, 1b, 3, 5, 6, 7, 8, 9 and 10 of the drawings, the cover member 40 includes at least one cover vent and preferably includes a plurality of cover vents 42. The cover vents 42 are comprised of a plurality of slots that extend radially outwardly from a central portion of the cover member 40 similar to the base vents 22 of the base member 20. The air is able to freely pass into the interior cavity of the housing by either/both the base vents 22 and/or the cover vents 42. The air is also able to freely pass out of the interior cavity of the housing by either/both the base vents 22 and/or the cover vents 42.

### E. Spacer Members.

The base member 20 preferably includes a plurality of spacer members 24 extending outwardly from a wall 28 of the base member 20 as illustrated in FIGS. 2, 4 through 8 and 10 of the drawings. The spacer members 24 extend outwardly from the outer surface of the wall 28 of the base member 20 sufficiently to allow for the free flow of air into and from the base vents 22 even when the base member 20 is adjacent to or touching an object (e.g. a building wall 11, a clothing item, the upper surface of a table 14). The spacer members 24 are preferably comprised of rounded nub structures that are positioned near the outer perimeter of the base member 20. It is preferable to have four spacer members 24 radially aligned approximately 90 degrees from adjacent spacer members 24 as best illustrated in FIG. 4 of the drawings.

### F. Housing Connectors.

The base member 20 and the cover member 40 each preferably include connectors 21, 41 that allow for selective connection of the base member 20 to the cover member 40. The base member 20 includes one or more base connectors 21 and the cover member 40 includes one or more cover connectors 41 as illustrated in FIGS. 9 and 10 of the drawings. The base connectors 21 are removably connectable to the cover connectors 41 to retain the cover member 40 connected to the base member 20. The base connectors 21 are each preferably comprised of an extended structure (e.g. a tongue member) extending inwardly from a base rim 26 of the base member 20 as illustrated in FIGS. 9 and 11 of the drawings. The cover connectors 41 are each preferably comprised of a slotted structure that receives one of the plurality of base connectors 21. The connectors 21, 41 may also be comprised of snaps, frictional fit and other connecting structures.

### G. Connecting Member.

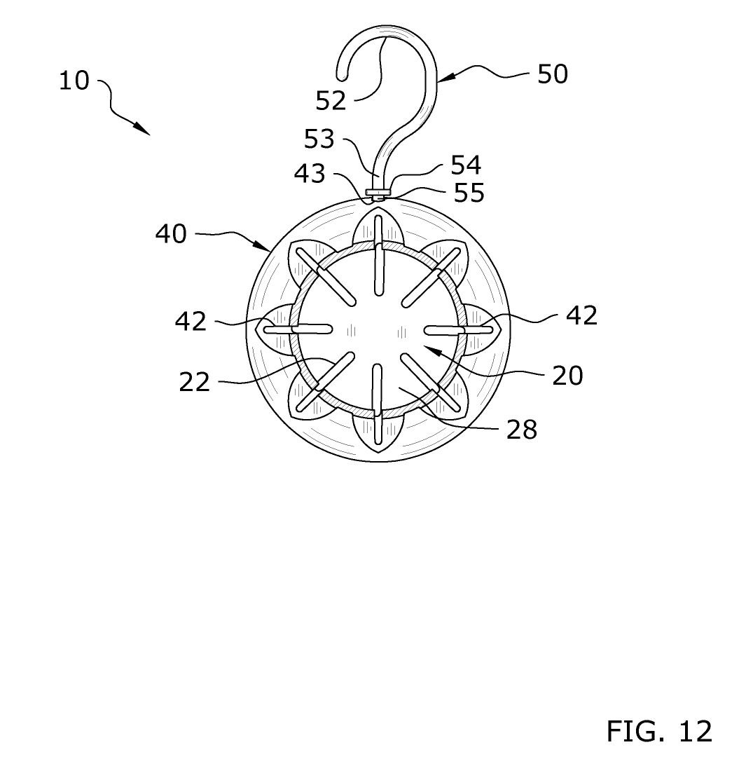

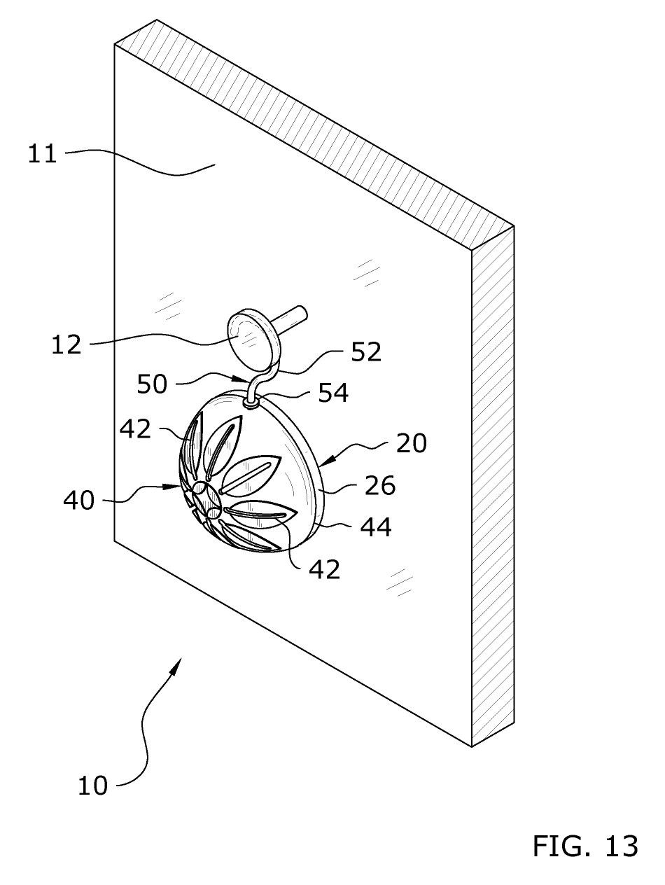

A connecting member 50 is removably connected to the housing so that the housing may be hung from an object (e.g. a fastener 12 extending from a building wall 11 as illustrated in FIG. 13 of the drawings). The connecting member 50 is preferably comprised of a hook 52 that has an open upper C-shaped end as illustrated in FIG. 12 of the drawings. The connecting member 50 further includes a lower portion 53 that is removably and catchably received within a connecting slot within the housing as illustrated in FIGS. 9 through 13 of the drawings.

The lower portion 53 of the connecting member 50 includes an upper guide member 54 and a lower guide member 56 defining a receiver slot 55 between thereof as best illustrated in FIGS. 9 through 11 of the drawings. When connected to the housing 20, 40, the upper guide member 54 is positioned externally of the housing and the lower guide member 56 is positioned internally of the housing with the receiver slot 55 receiving the upper portion of the housing (a portion of the base member 20 and a portion of the cover member 40 as illustrated in FIG. 11 of the drawings).

The connecting slot within the housing preferably extends into either the distal portion of the cover member 40 (see FIGS. 9 and 10) or the base member 20 (not illustrated in the drawings). The connecting slot may extend into both of the cover member 40 and the base member 20 to removably receive the connecting member 50. The connecting slot is preferably comprised of a cover slot 43 extending inwardly from an outer perimeter of the cover member 40 as illustrated in FIGS. 9 and 10 of the drawings.

### H. Operation of Preferred Embodiment.

In use, the user positions the fragrance dispenser 30 within the housing to be formed by the base member 20 and the cover member 40. If the invention is to be hung from an object such as a fastener 12, the user positions the connecting member 50 into the cover slot 43 within the cover member 40 prior to attaching the cover member 40 to the base member 20. The user then secures the cover member 40 to the base member 20 with the connecting member 50 attached between the base member 20 and the cover member 40 as best illustrated in FIG. 11. The user then is able to attach the connecting member 50 to a fastener 12 or other object to hang the housing with the fragrance dispenser 30 to dispense the fragrance by air flowing through the cover vents 42 and the base vents 22.

Alternatively, if the user intends to use the invention by sitting the housing upon a surface such as the upper surface of a table 14, the user does not insert the connecting member 50 into the cover slot 43 (or removes the connecting member 50 if already in the cover slot 43) prior to connecting the cover member 40 to the base member 20. The user then positions the present invention upon the upper surface of the table 14 with the spacer members 24 supporting the wall 28 of the base member 20 above the upper surface of the table 14 to allow for airflow through the base vents 22 upwardly through the cover vents 42.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. An scent dispenser housing, comprising:
a housing comprised of a base member and a cover member;
wherein said base member includes at least one base vent; and
wherein said cover member includes at least one cover vent, wherein said cover member is removably connected to the base member to form an interior cavity within said housing that receives a fragrance dispenser and wherein air is allowed to freely flow through said at least one cover vent through said interior cavity then through said at least one base vent.

2. The scent dispenser housing of claim 1, wherein said base member includes a plurality of spacer members extending outwardly from a wall of said base member.

3. The scent dispenser housing of claim 2, wherein said at least one base vent is comprised of a plurality of base vents.

4. The scent dispenser housing of claim 3, wherein said plurality of base vents are comprised of a plurality of slots.

5. The scent dispenser housing of claim 4, wherein said plurality of slots extend radially outwardly from a central portion of said wall.

6. The scent dispenser housing of claim 1, wherein said cover member has a dome shape.

7. The scent dispenser housing of claim 1, wherein said cover member is rotatably connected to and rotatably removed from said base member.

8. The scent dispenser housing of claim 1, wherein said base member includes a plurality of base connectors and wherein said cover member includes a plurality of cover connectors, wherein said plurality of base connectors removably connect to said plurality of cover connectors to retain said cover member connected to said base member.

9. The scent dispenser housing of claim 8, wherein said plurality of base connectors are each comprised of an extended structure extending inwardly from a base rim of said base member and wherein each of said plurality of cover connectors are comprised of a slotted structure that receives one of said plurality of base connectors.

10. The scent dispenser housing of claim 9, wherein said base member includes a wall that said base rim extends forwardly from, wherein said wall and said base rim are circular shaped, and wherein said wall and said base rim define a base cavity.

11. The scent dispenser housing of claim 10, wherein said cover member defines a cover cavity, wherein said base cavity and said cover cavity together form said interior cavity when said cover member is connected to said base member.

12. The scent dispenser housing of claim 1, wherein said base member includes a base rim and wherein said base rim includes a base recessed portion within an outer perimeter of said base rim to receive a corresponding shaped outer perimeter of said cover member.

13. The scent dispenser housing of claim 12, wherein said corresponding shaped outer perimeter of said cover member includes a cover recessed portion.

14. The scent dispenser housing of claim 13, wherein said cover recessed portion is on a side of said cover member opposite of a side of said base rim of said base member.

15. The scent dispenser housing of claim 1, wherein said at least one cover vent is comprised of a plurality of cover vents, wherein said plurality of cover vents are comprised of a plurality of slots and wherein said plurality of slots extend radially outwardly from a central portion of said cover member.

16. The scent dispenser housing of claim 1, including a connecting member removably connected to said housing.

17. The scent dispenser housing of claim 16, wherein said connecting member is comprised of a hook.

18. The scent dispenser housing of claim 16, wherein said connecting member includes a lower portion that is removably and catchably received within a connecting slot within said housing.

19. The scent dispenser housing of claim 18, wherein said connecting slot is comprised of a cover slot extending inwardly from an outer perimeter of said cover member.

20. The scent dispenser housing of claim 16, wherein said lower portion includes an upper guide member and a lower guide member defining a receiver slot between thereof, wherein said upper guide member is positioned externally of said housing and wherein said lower guide member is positioned internally of said housing when said connecting member is connected to said housing.
